(19) United States
(12) Plant Patent Application Publication
(10) Pub. No.: US 2003/0237120 P1
(43) Pub. Date: Dec. 25, 2003

(54) Mandarin hybrid tree named "TDE4"

(75) Inventors: Mikeal L. Roose, Riverside, CA (US); Timothy A. Williams, Riverside, CA (US); Robert K. Soost, Inverness, CA (US); James W. Cameron, Salem, OR (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California Office of Technology Transfer University of California, Oakland, CA (US)

(21) Appl. No.: 10/177,417

(22) Filed: Jun. 20, 2002

(51) Int. Cl. A01H 5/00

(52) U.S. Cl. PLT/202

(57) ABSTRACT

A new mandarin hybrid called “TDE4” is distinguished by production of fruit that combine mid-late season maturity, large fruit size, attractive deep orange rind color and virtual absence of seeds with rich fruit flavor.

### BACKGROUND OF THE INVENTION

The pedigree of TDE4 is shown in FIG. 1. In 1973, pollen from Encore mandarin was applied to stigmas of a tetraploid (Temple×4N Dancy) hybrid and the pollinated flowers were bagged to prevent insect pollination. Fruits were collected in winter 1974, seeds extracted from each fruit, and each seed was planted. The chromosome number of each seedling was determined and those identified as triploid seedlings were budded onto Troyer rootstock. The resulting trees were planted in the field in Riverside, Calif. in 1976. These trees were evaluated for tree vigor, bearing, and seediness, fruit flavor, fruit color, and other fruit quality traits from bearing until 1985. Five trees were selected from the original population and repropagated by budding onto C-32 citrange, C-35 citrange, Troyer citrange, and trifoliate orange rootstocks. Two trees of the selection now called TDE4 were planted in the field in Riverside in 1987. When they began fruiting (approximately in 1990), these trees were evaluated for the same tree and fruit quality traits as the original trees. In 1987, the selection now called TDE4 was chosen for additional testing because it combined medium or large fruit size, low seed number, rich fruit flavor, deep orange rind and flesh color, and acceptable peelability. Budwood of this selection was tested for viruses and other pathogens by the Citrus Clonal Protection Program and virus-free bud source trees were planted at Lindcove Research and Extension Center, Exeter, Calif. in 1991.

Using this virus-free budwood source, additional trees were propagated and planted at several California locations between 1993 and 1996. These included one location in the Coachella Valley (the Coachella Valley Agricultural Research Station-CVARS, 8 trees), Ojai (12 trees) and Santa Paula (5 trees) in Ventura Co., and two locations in the San Joaquin Valley, (Lindcove Research and Extension Center, 8 trees, and Orange Cove, 8 trees). These trial plantings provide most of the available data on TDE4. Several different rootstocks have been used in these evaluations, including Carrizo citrange, C35 citrange, Rich 16-6 trifoliate, Cleopatra mandarin, and Schaub rough lemon. In general, no major effects of these rootstocks on fruit quality of TDE4 were observed, and no incompatibilities have been evident, but longevity of trees on various rootstocks is not known. Effects of rootstocks on tree size are discussed below.

### BRIEF SUMMARY OF THE INVENTION

The present invention provides a novel mandarin hybrid having the characteristics described and illustrated herein. The hybrid TDE4 produces fruit that combine mid-late season maturity, large fruit size, attractive deep orange rind color and virtual absence of seeds with rich fruit flavor.

### BRIEF DESCRIPTION OF THE DRAWINGS

FIG. 1 illustrates the pedigree of TDE4. All cultivars are C. reticulata except orange, which is C. sinensis.

FIG. 2 illustrates, clockwise from top left: a nine-year-old tree of TDE4 on Carrizo rootstock; fruit on tree; branching pattern; flower buds; flowers; leaves; and shoots.

FIG. 3 illustrates fruit of TDE4 sampled from nine-year-old tree on Carrizo rootstock.

FIGS. 4A-D illustrate the solids:acid ratio of TDE4 at four California locations over five years. Points plotted are means of all samples collected on a given date. Solid lines connect means for sampling dates within the same season. The dashed line is a liner regression of solids:acid on sampling date using data from all years. The regression equation and r² value are shown in each figure.

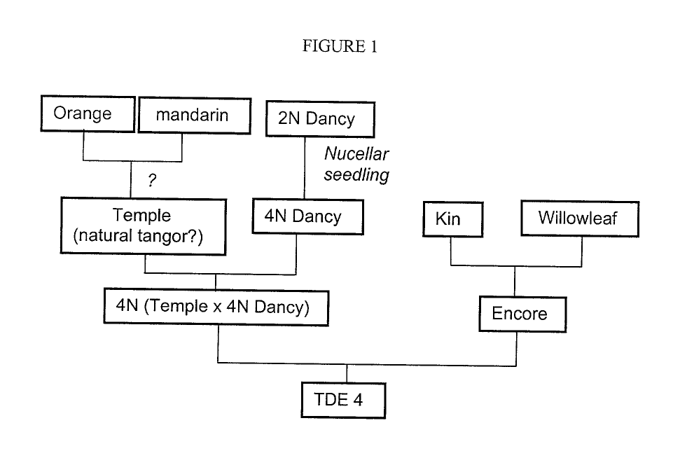

### DETAILED DESCRIPTION OF THE INVENTION

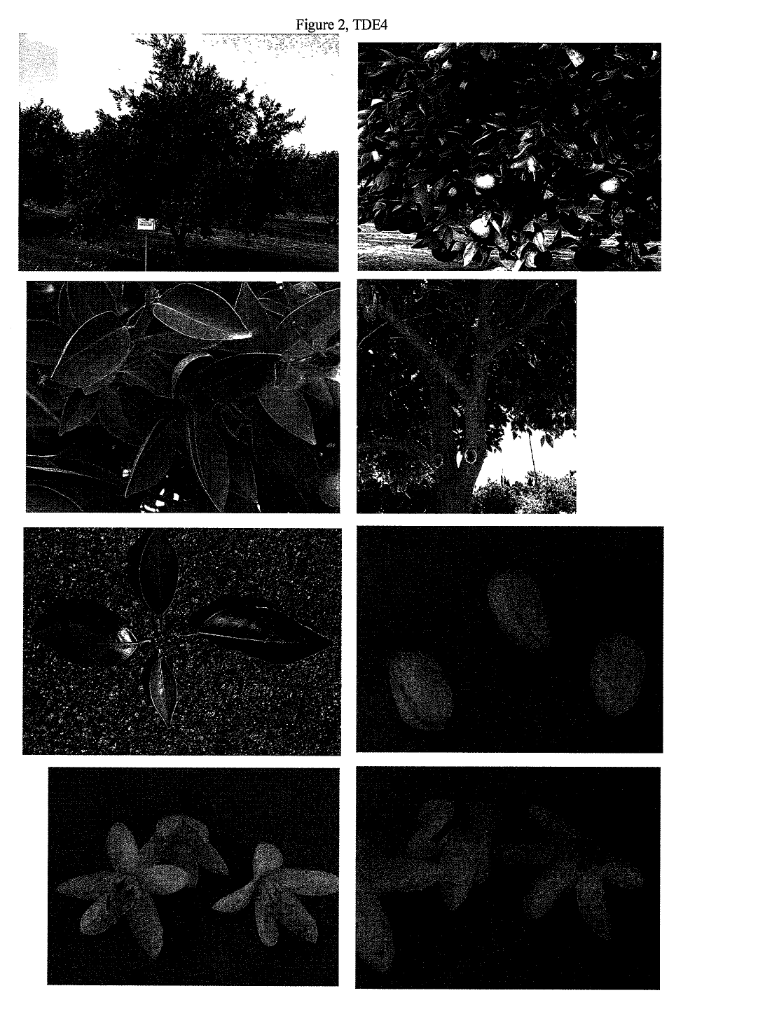

Tree shape (FIG. 2) is approximately sphereoid, rather similar to that of orange trees. The trees have not been noted as particularly susceptible to any diseases and, based on a freeze in 1999, appeared only slightly more cold hardy than oranges of similar age. Leaves (FIG. 2) are simple, brevipetiolate, lanceolate, with entire or slightly sinuate margins. The petiole shape is narrow and linear in shape. In comparison with most old-line citrus cultivars, trees of TDE4 are somewhat thorny, with normal branches having short length (4 mm) thorns at about 13% of the nodes, and vigorous sprouts having short (3 mm) thorns at about 3% of nodes. Thorniness will probably decrease as the cultivar ages.

Flowers of TDE4 are typically hermaphroditic, with white petals and yellow anthers (FIG. 2). Trees flower from early April into May at most locations. Pollen is somewhat sparse, with viability (estimated in an in vitro germination test) of 8%. Pollen tube growth is also less than that of fertile, diploid mandarins.

If sufficient fruit was available, 10-fruit samples were collected from each location two or three times each year beginning in 1997 or 1998. Generally samples were collected from two or three trees per location on each sampling date. These fruit were evaluated in Riverside for a range of traits as summarized in Table 1.

TABLE 1
Fruit characteristics of TDE4 averaged over 4 locations and 4 seasons.
Samples were collected from mid-January to early May at Santa Paula,
Ojai, and Lindcove, and from mid-January to mid-March at Orange
Cove. “N” indicates the total number of fruit samples analyzed.
Results are averaged over several rootstocks.

| Trait | N | Min | Max | Mean | SD |
|---|---|---|---|---|---|
| Fruit height (mm) | 201 | 47.5 | 76.8 | 58.3 | 5.29 |
| Fruit width (mm) | 201 | 59.6 | 102.1 | 74.7 | 7.15 |
| Fruit height:width | 201 | 0.67 | 0.91 | 0.78 | 0.045 |
| Rind color | 201 | 4.5 | 13.0 | 12.3 | 1.04 |
| Rind texture | 201 | 2.3 | 5.0 | 3.3 | 0.57 |
| Neck | 201 | 0 | 2.00 | 0.23 | 0.430 |
| Peelability | 201 | 5.00 | 10.00 | 8.23 | 0.850 |
| Rind thickness (mm) | 201 | 3.00 | 6.00 | 4.21 | 0.687 |
| Seeds per fruit | 201 | 0 | 5.00 | 0.32 | 0.696 |
| Fruit weight (g) | 201 | 91.0 | 335.0 | 174.5 | 41.94 |
| Juice content (%) | 195 | 18.2 | 56.3 | 42.2 | 6.91 |
| Soluble solids (%) | 195 | 7.85 | 19.50 | 12.31 | 1.698 |
| Acid (%) | 195 | 0.56 | 2.03 | 1.13 | 0.302 |
| Solids:acid | 195 | 5.81 | 24.60 | 11.61 | 3.332 |

^{a}Visual rating on a scale of 0-13; 0 = green, 13 = red-orange
^{b}Visual rating on a scale of 1-8; 1 = very smooth, 8 = extremely coarse
^{c}Visual rating on a scale of 0-3; 0 = no trace of neck, 3 = neck with a diameter at least 50% of fruit diameter
^{d}Subjective rating of ease of peeling a single fruit; 1 = very difficult, 10 = a fruit with completely separated rind and segments. Fruit with ratings of 7 or higher would be relatively easy to peel.

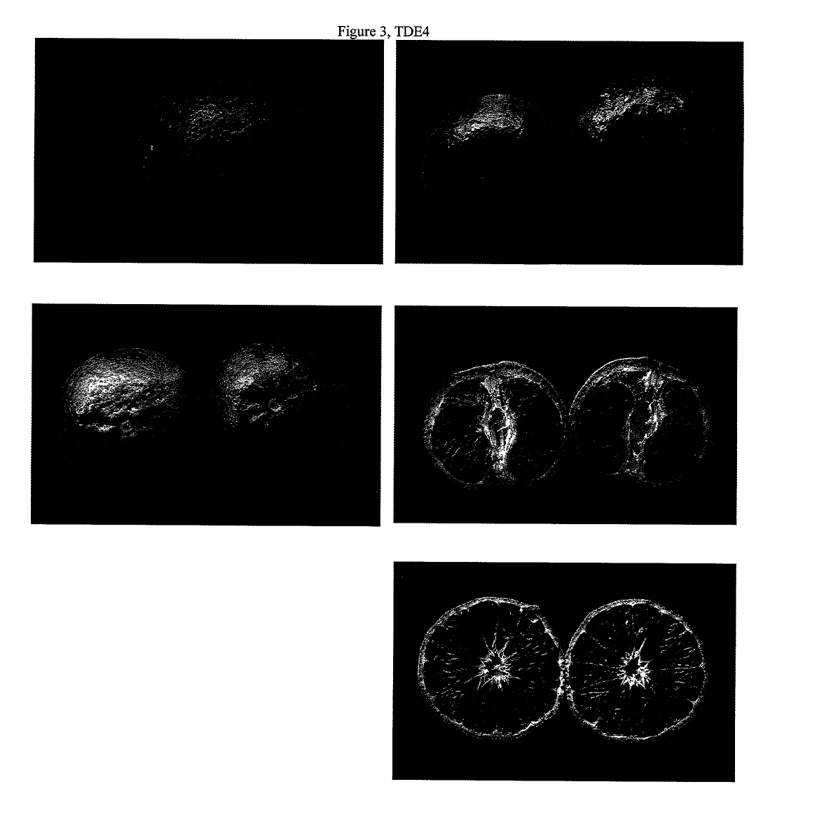

Based on this data, TDE4 fruit are oblate in shape (FIG. 3), with little or no neck. The average fruit size is large for a mandarin (classed as Mammoth by California state standards). Rind color is deep orange with color scores of L=63. 1, C=65.9, H=62.4 for fruit harvested in Riverside on February 10. The rind texture is somewhat variable, depending on tree age and crop. For older trees with a moderate to heavy crop, rind texture is smooth, with conspicuous oil glands (about 50 cm²). The rind of fruit from trees with very light crops is sometimes excessively rough or bumpy. The fruit base (stalk end) is slightly concave (FIG. 3), and the apex is truncate with a slight depression in the stylar end and a small (2 mm), usually closed, stylar scar. The rind is quite easy to peel when fruit are mature, but can be more adherent early in the season.

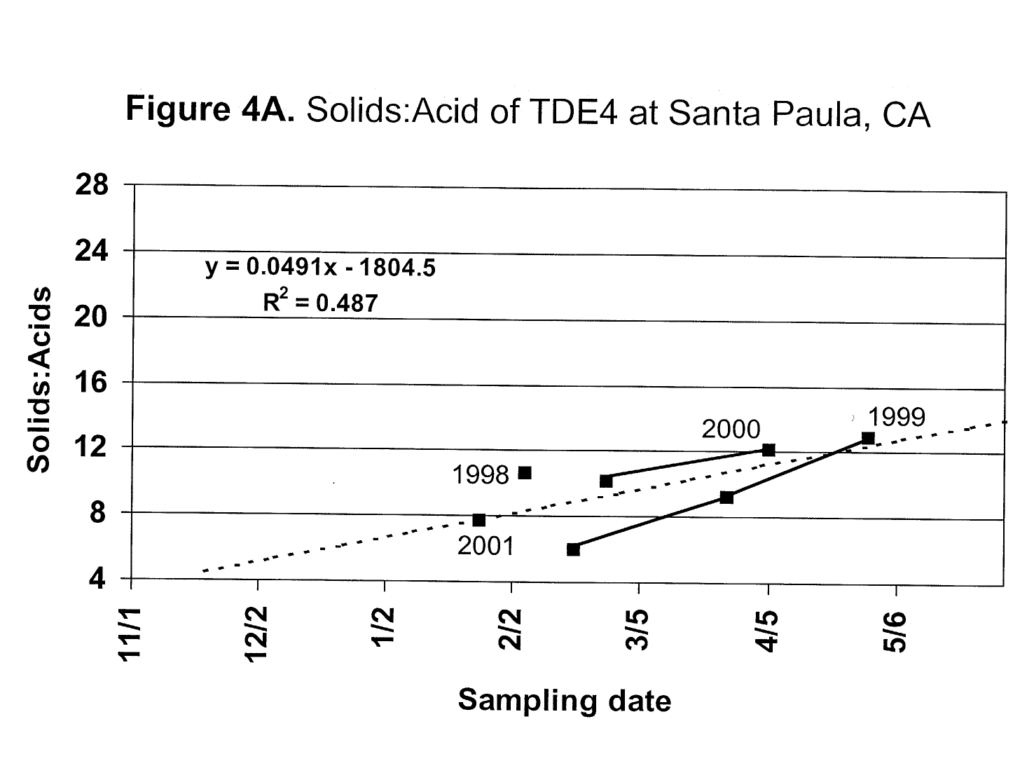

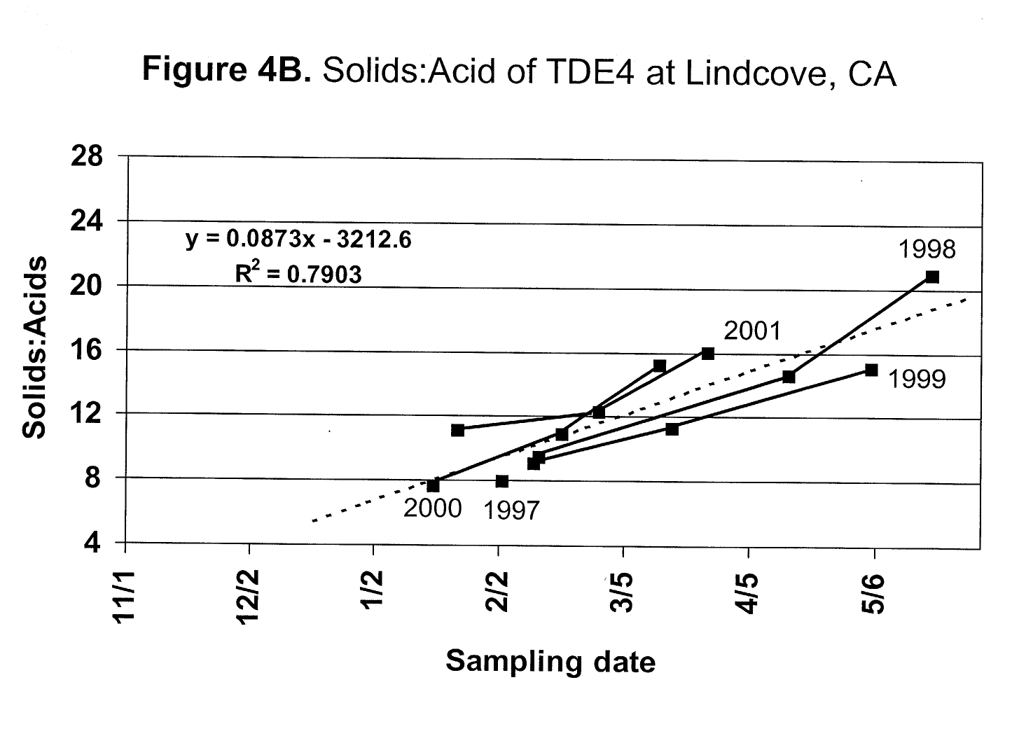

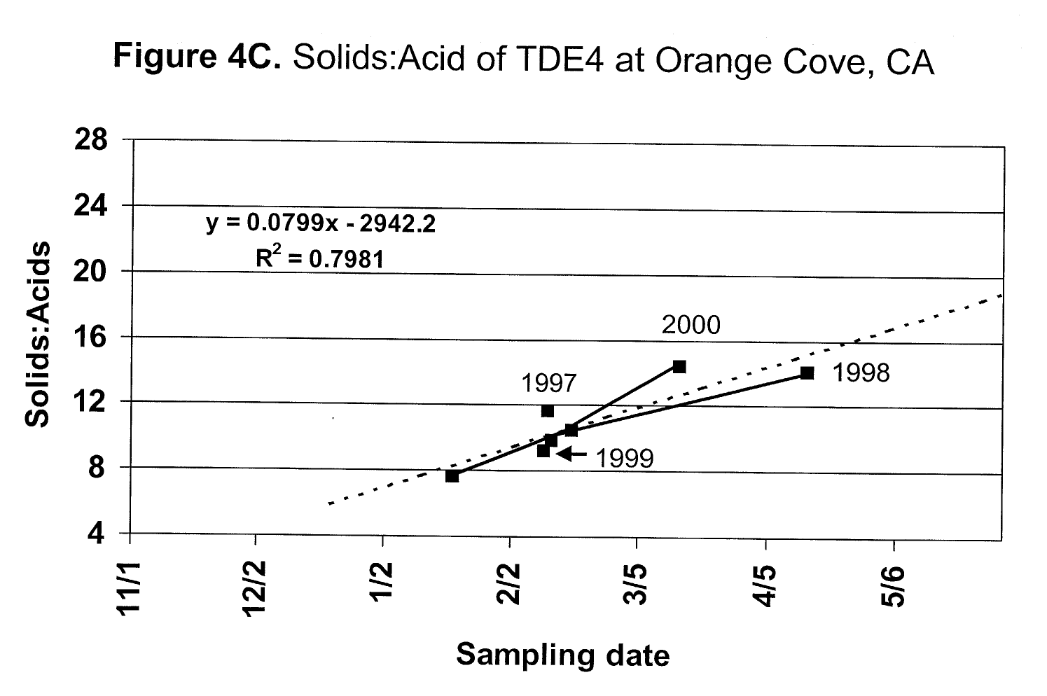

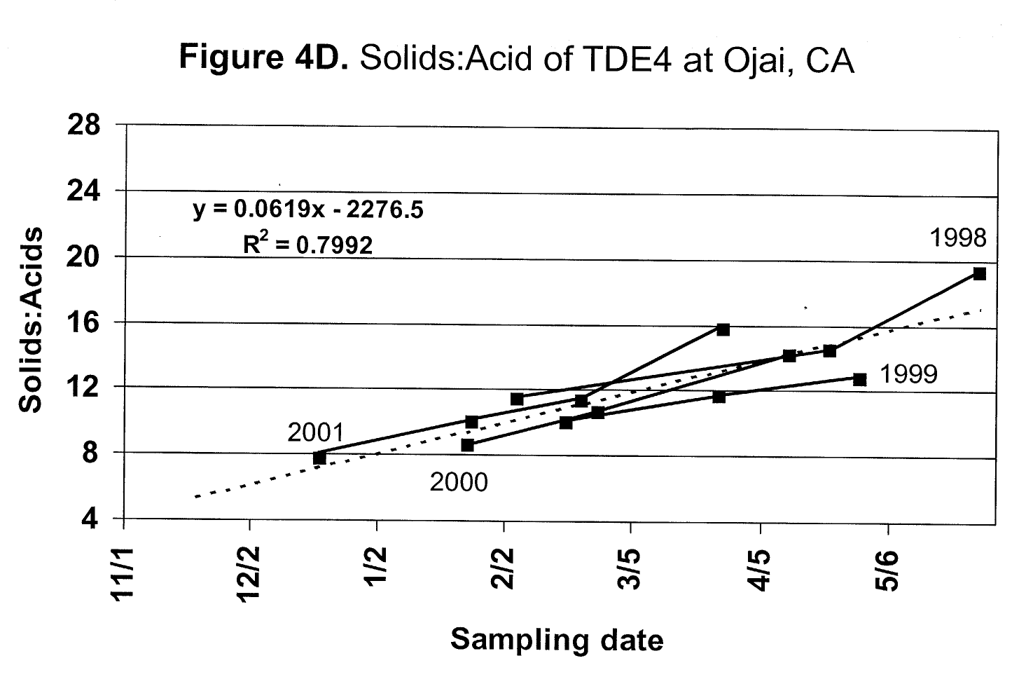

Important determinants of maturity date for citrus fruit are the solids:acid ratio and juice content. Using data for all years, juice content show a statistically significant correlation with sampling date at only at Santa Paula, where the slope of the regression was positive. Regressions were slightly negative at the other three locations, but not significantly so. This indicates that at Santa Paula, the site with the latest maturity date, fruit sampled from mid-January to mid-February had not yet reached maturity. At the other locations, juice content showed little tendency to decrease later in the season. Solids:acids ratio was significantly correlated with sampling date at all location except Santa Paula (FIG. 4). Using these regressions, the estimated dates on which fruit reached an 8:1 solids:acid ratio was January 2 for Ojai, January 15 for Orange Cove, January 16 for Lindcove, and January 27 for Santa Paula.

During the 1999-2000 season, fruit of TDE4 and several other mandarin varieties were harvested, run over a packline at the University of California Lindcove Research and Extension Center, waxed and evaluated by a taste panel. Evaluations were done before storage, after storage for 11 days at 68 F., and after storage for 21 days at 37 F. Fruit were rated on a 9 point scale, where a score of 1 is “Dislike extremely”, 5 is “Neither dislike or like”, and 9 is “Like extremely”. Fruit were sampled from test plots at Lindcove and Orange Cove on February 23 (Table 2) and Mar. 21, 2000 (Table 3). These samples would represent mid-late season fruit of TDE4, the fruit from Lindcove and Orange Cove having solids:acid ratios of 10.8 and 10.5 on February 18 and 15.1 and 14.3 on March 14 respectively. TDE4 fruit from the two locations were similar in all traits evaluated. Their ratings were good for all traits before storage, and were little changed by storage at room temperature or at 37 F. TDE4 had higher scores than Gold Nugget and W. Murcott for visual appeal and similar peelability. It also had slightly higher taste scores in most comparisons.

TABLE 2
Sensory panel evaluation of TDE4 (TDE4L), Gold Nugget, W. Murcott from Lindcove and TDE4 from
Orange Cove (TDE4M) harvested February 22, 2000.

| | | Visual Evaluation | | | | Peelability Evaluation | | | | Taste Evaluation | | | |
|---|---|---|---|---|---|---|---|---|---|---|---|---|---|
| | | | | Gold | W. | | | Gold | W. | | | Gold | W. |
| Storage | | TDE4L | TDE4M | Nugget | Murcott | TDE4L | TDE4M | Nugget | Murcott | TDE4L | TDE4M | Nugget | Murcott |
| Initial | Mean | 7.0 | 7.2 | 3.6 | 5.3 | 7.5 | 6.8 | 7.2 | 6.6 | 7.3 | 7.2 | 6.5 | 6.2 |
| | SD | 1.6 | 1.4 | 1.6 | 1.6 | 0.8 | 2.2 | 1.5 | 2.1 | 1.1 | 1.7 | 1.6 | 1.9 |
| 11 days | Mean | 7.2 | 7.3 | 6.7 | 5.3 | 6.7 | 7.2 | 8.1 | 7.5 | 6.1 | 5.9 | 5.5 | 6.9 |
| @ 68 F. | | | | | | | | | | | | | |
| | SD | 1.1 | 1.3 | 1.2 | 2.0 | 1.6 | 1.7 | 1.3 | 1.7 | 2.5 | 1.9 | 2.1 | 2.5 |
| 21 days | Mean | 7.2 | 7.5 | 6.7 | 4.2 | 7.1 | 7.6 | 8.0 | 7.0 | 6.7 | 6.6 | 5.7 | 6.9 |
| @ 37 F. | | | | | | | | | | | | | |
| | SD | 1.6 | 1.7 | 2.2 | 1.8 | 1.7 | 1.9 | 1.8 | 2.0 | 2.1 | 1.9 | 1.9 | 2.1 |

TABLE 3
Sensory panel evaluation of TDE4 (TDE4L), Gold Nugget, W. Murcott from Lindcove and TDE4 from
Orange Cove (TDE4M) harvested Mar. 20, 2000.

| | | Visual Evaluation | | | | Peelability Evaluation | | | | Taste Evaluation | | | |
|---|---|---|---|---|---|---|---|---|---|---|---|---|---|
| | | | | Gold | W. | | | Gold | W. | | | Gold | W. |
| Storage | | TDE4L | TDE4M | Nugget | Murcott | TDE4L | TDE4M | Nugget | Murcott | TDE4L | TDE4M | Nugget | Murcott |
| Initial | Mean | 6.8 | 7.1 | 4.5 | 6.6 | 6.9 | 7.2 | 7.7 | 7.8 | 7.5 | 6.9 | 7.1 | 6.8 |
| | SD | 1.6 | 1.7 | 1.3 | 1.4 | 1.8 | 1.9 | 1.0 | 0.9 | 1.2 | 1.4 | 1.7 | 1.9 |
| 11 days | Mean | 6.9 | 7.4 | 5.8 | 7.3 | 6.6 | 7.3 | 7.6 | 7.6 | 7.0 | 6.8 | 6.7 | 6.7 |
| @ 68 F. | | | | | | | | | | | | | |
| | SD | 1.5 | 1.5 | 1.2 | 0.9 | 1.6 | 1.4 | 1.5 | 1.3 | 2.1 | 1.3 | 1.8 | 1.7 |
| 21 days | Mean | 6.8 | 7.5 | 6.9 | 5.3 | 7.0 | 7.7 | 7.8 | 7.1 | 7.4 | 6.7 | 6.3 | 6.6 |
| @ 37 F. | | | | | | | | | | | | | |
| | SD | 2.1 | 1.1 | 1.4 | 2.2 | 1.6 | 1.2 | 1.4 | 1.8 | 1.8 | 1.4 | 1.8 | 1.7 |

Yield of TDE4 was evaluated from visual ratings of crop relative to tree size at each location from 1998-99 to 2001-2002. The rating scale ranged from 0 (no crop) to 5 (very heavy crop). Crops at Ojai were fairly good, being 2-3.3 during the last three of the four years evaluated. At Santa Paula, crop ratings indicated moderate alternate bearing, with average values of 0.50, 2.60, 0.88, and 2.90 from 1998-99 to 2001-2002 respectively. Trees planted at Lindcove in 1994 showed similar behavior, 2.94, 1.88, 1.50, and 2.90 from 1998-99 to 2001-2002 respectively. At Orange Cove, trees showed rather severe alternate bearing with crop ratings of 1.88, 4.00, 0.06, and 1.60. Yield at Lindcove in 2000 and 2001 was 29 and 14 kg tree⁻¹, while at Orange Cove it was 66 and 0 kg tree⁻¹. Trees appear to flower profusely, but fruit set is virtually absent.

Trees that were screened to exclude bees during flowering produced very few fruit for two consecutive years, but it is possible that TDE4 is self-fertile but requires pollination for fruit set. Because TDE4 is a mid-late season fruit, it is likely that trees will show a fairly strong tendency to alternate bearing, and this is supported by the data for some locations.

Two siblings of TDE4, “TDE2” and “TDE3,” were compared to TDE2. TDE4 is distinct from these cultivars in having a smoother rind, intermediate maturity date, and distinctive flavor. TDE4 fruit are more oblate in shape than those of TDE3, and the rind color of TDE4 is deeper orange than that of TDE2. Trees or fruit of TDE4 can be distinguished from those of other mandarins, including TDE2 and TDE3, using simple sequence repeat (SSR) DNA markers. Using TDE4 DNA as template, PCR primer set TAA3 (F=AGAGAAGAAACATTTGCGGAGC, R=GAGATGGGACTTGGTTCATCACG) amplified a band of 145 bp while TDE2 and TDE3 had both had two bands of 142 and 145 bp. Primer sets TAA3 plus CAC15 (F=TAAATCTCCACTCTGCAAAAGC, R=GATAGGAAGCGTCGTAGACCC) and TAA15 (F=GAAAGGGTTACTTGACCAGGC, R=CTTCCCAGCTGCACAAGC) distinguished TDE4 from the following cultivars: Dancy, Encore, King, Willowleaf, Wilking, Gold Nugget, Pixie, W. Murcott, Ellendale, Hernandina Clementine, Fortune, Kara, Kinnow, Murcott, Nova, and Ponkan.

Vigor of TDE4 trees has varied greatly across locations. At CVARS, where the trees grew rapidly, canopy volumes of 7-year-old trees averaged 23.0 m³. In contrast, at the cooler Santa Paula and Ojai locations, 7-year-old trees averaged 4.3 and 5.6 m³. Trees in the desert locations have never produced fruit, perhaps contributing to greater vegetative growth. Rootstocks affected tree size at some locations. At Lindcove and Orange Cove, trees on Carrizo were the largest, followed by C35, and then Cleo and trifoliate which were similar. At Ojai, the largest trees were on C35, followed by Schaub rough lemon and Carrizo. At CVARS, trees on Carrizo, C35 and Cleo were similar in size. At Santa Paula, the single tree on Carrizo was smaller than that on C35. No evidence of rootstock-scion incompatibilities was evident.

TDE4 can be propagated on many available citrus rootstocks by budding. To reduce thorniness, budwood should be selected from thornless, upper canopy branches. Tree spacing in field plantings will depend on vigor of the rootstock. For Carrizo citrange rootstocks, a recommended tree density is about 150 trees per acre. Higher densities are possible, but will require more frequent pruning or hedging. Care of young trees should be similar to that used for other mandarins or oranges. Trees can be grown with pollinizer cultivars such as Minneola, Valencia orange, or unrelated mandarins (not Temple, Dancy, Encore or other TDE hybrids) that produce viable pollen. Optimal pruning practices have not yet been developed, but in many locations trees will perform well with relatively little pruning. Maturity dates will vary with location, probably depending on the number of heat units and soil conditions.

As with some other mandarins, sprays with gibberellic acid may increase fruit set when pollinizers and/or pollinators are inadequate.

What is claimed is:

1. A new and distinct variety of mandarin hybrid tree having substantially the characteristics described and illustrated herein.
